(19) United States
(12) Patent Application Publication
(10) Pub. No.: US 2002/0150537 A1
(43) Pub. Date: Oct. 17, 2002

(54) Method for assessing food allergenicity

(76) Inventors: Gregorio del Val, San Diego, CA (US); Boihon C. Yee, Walnut Creek, CA (US); Hye Rim Jung, Alameda, CA (US); Bob Buchanan, Berkeley, CA (US); Oscar L. Frick, San Francisco, CA (US)

Correspondence Address: MORRISON & FOERSTER LLP, 425 MARKET STREET, SAN FRANCISCO, CA, 94105-2482, US

(21) Appl. No.: 09/776,454

(22) Filed: Feb. 2, 2001

(51) Int. Cl. A61K 49/00

(52) U.S. Cl. 424/9.2; 800/3

(57) ABSTRACT

A method for testing the allergenicity of a heterologous protein produced by a plant or animal that has been genetically modified to produce that protein is disclosed. The method includes the steps of: (a) sensitizing a newborn dog from an atopic dog colony with a first extract prepared from tissue of the genetically modified plant or animal and containing a mixture of plant or animal proteins and the heterologous protein, by injecting or feeding the extract into the newborn dog; (b) after a period sufficient to allow the dog to establish an immune response to the sensitizing extract, challenging the dog with the extract; (c) observing the degree of allergic response provoked; (d) if a detectable skin reaction is observed, comparing the degree of skin reaction observed with that observed by carrying out steps (a)-(c) above, but where the sensitizing step (a) or applying step (b) is carried out with a second plant or animal extract containing substantially the same proteins as the first extract but lacking the heterologous protein; and (e) if the degree of skin reaction at (c) is greater than that observed by carrying out steps (a)-(c) in accordance with step (d), identifying the heterologous protein as a potential allergen in humans. Also disclosed is a dog for use in testing a biological substance for allergenicity in humans, and compositions useful in practicing the method.

This invention was funded by a grant from the USDA, Grant No. CA-B-PLM-6163-H. The government has certain rights in the invention.

### FIELD OF THE INVENTION

The present invention relates to methods for identifying allergens in genetically modified organisms, and to animal models and compositions useful in practicing the method.

References

Ausubel, et al., (c) 1987, 1988,1989, 1990, 1993, CURRENT PROTOCOLS IN MOLECULAR BIOLOGY, John Wiley & Sons, New York, N.Y.

Barboza, D (2000) Gene-Altered Corn Changes Dynamics Of Grain Industry. New York Times December 11.

Bischoff, S. C., (1997) Internat. Arch. Allergy and Immunol. 113: 348-351.

Bischoff, S. C., (1997) Gut 40: 745-753.

Bradford, M. A. (1976) A rapid and sensitive method for the qualification of microgram quantities of protein utilizing the principle of protein-dye binding. Analytical Biochemistry 72:248-254.

Buchanan, B. B., Adamidi, C., Lozano, R. M., Yee, B. C., Momma, M., Kobrehel, K., Ermel, R., & Frick, O. L. (1997) Thioredoxin-linked mitigation of allergic responses to wheat. Proc. Natl. Acad. Sci. USA 94:5372-5377.

Chung, K. F., Aizawa, H., Becker, A. B., Frick, O., Gold, W. M. & Nadel, J. A. (1986). Inhibition of antigen-induced airway hyperresponsiveness by a thromboxane synthetase inhibitor (OKY-046) in allergic dogs. Amer. Rev. Resp. Dis. 134:258-261.

Chung, K. F., Becker, A. B., Lazarus, S. C., Frick, O. L., Nadel, J. A. & Gold, W. M. (1985). Antigen-induced airway hyperresponsiveness in pulmonary inflammation in allergic dogs. J. Appl. Physiol. 58: 1347-53.

del Val, G., Yee, B. C., Lozano, R. M., Buchanan, B. B., Ermel, R. W., Lee, Y. M. & Frick, O. L. (1999) Thioredoxin treatment increases digestibility and lowers allergenicity of milk. J. Allerg. Clin. Immunol. 104: 690-697.

Ermel, R. W., Kock, M., Griffey, S. M., Reinhart, G. A. & Frick, O. L. (1997) The atopic dog: A model for food allergy. Laboratory Animal Science 47:40-49.

Frick, O. L., Gertz, E., Wong, J. H., del Val, G., Yee, B. C., Buchanan, B. B., Petersen, W. R., Teuber, S. S. & Ross, R. (1999) New evidence strengthening the dog as a model for human allergies. Amer. Acad. Allergy Asthma. Immunol. 1999 Annual Meeting, Orlando, Fla. J. Allergy Clin. Immunol. 103: S98.

Gelvin, S. B., et al., (1990) eds. PLANT MOLECULAR BIOLOGY MANUAL.

Gordon (1989) Transgenic Animals. Intl. Rev. Cytol. 115: 171-229.

Gordon et al. (1980) Proc. Natl. Acad. Sci. USA 77: 7380-7384.

Gordon and Ruddle (1981), Science 214: 1244-1246.

Hinswehr, R., Heppner, C., Spitzauer, S., Sperr, W. R., Valent, P., Berger, U., Horak, F., Jäger, S., Kraft, D. & Valenta, R. (1998) Allergens, IgE, mediators, inflammatory mechanisms: Identification of common allergenic structures in mugwort and ragweed pollen. J. Allerg. Clin. Immunol. 101: 196-206.

Kobrehel, K., Wong, J. H., Balogh, A., Kiss, F., Yee, B. C. and Buchanan, B. B. (1992) Specific reduction of wheat storage proteins by thioredoxin h. Plant Physiol. 99, 919-924.

Lavitrano et al. (1989) Cell 57: 717-723.

Lee, C. -M., Changschien, C. -S., Chen, P. -C., Lin, D. -Y., Sheen, I. S., Wang, C. -S., Tai, D. -I., Sheen-Chen, S. -M., Chen, W. -S. & Wu, C. -S. (1993) Eosinophilic gastronententeritis: 10 years experience. Amer. J. Gastroenterol 88: 70-74.

Lo (1983) Mol. Cell Biol. 3: 1803-1814.

Sambrook, et al., (1989) MOLECULAR CLONING: A LABORATORY MANUAL, 2d Edition.

Niederberger, V., Pauli, G., Grönlund, Fröschl, R., Rumpold, H., Kraft, D., Valenta, R. and Spitzauer, S. (1998) Recombinant birch pollen allergens (rBet v1 and rBet v2) contain most of the IgE epitopes present in birch, alder, hornbeam, hazel and oak pollen: A quantitative IgE inhibition study with sera from different populations. J Allergy Clin Immunol. 102:579-91.

Taylor, S. L., (1997) “Assessment of the allergenicity of genetically modified foods”, AgBiotech-News-and-Information 9: 10, 229N-234N; 40.

Thompson et al. (1989) Cell 56: 313-321.

Valenta, R., Duchene, M., Christof, E., Valent, P., Sillaber, C., Deviller, P., Ferreira, F., Teijkl, M., Edelmann, H., Kraft, D. & Scheiner, O. (1992) Profilins constitute a novel family of functional plant pan-allergens. J. Exp. Med. 175: 377-385.

Van der Putten et al. (1985) Proc. Natl. Acad. Sci. USA 82: 6148-6152.

### BACKGROUND OF THE INVENTION

Genetic modification through biotechnology allows an organism to produce a particular trait, i.e., express a protein or other characteristic, which is novel for that organism. A particular issue raised by critics and other observers of the debate about genetically modified foods relates to new proteins produced in genetically engineered foods, with the expressed concern that these could act as allergens, either by themselves or in unintended ways in humans. In the case of a heterologous protein, allergenicity may be due to processing of proteins (e.g., glycosylation by the transgenic organism), or may be due to native proteins in the transgenic environment.

This concern derives from the fact that genetic engineering is different from traditional breeding, in that genetic engineering can move genetic material between completely unrelated plant species, and even between the plant, animal and microbe kingdoms in ways that could never occur in nature. Thus, even for proteins not known to be allergenic, there are concerns that when expressed in a genetic construct in a new organism, the proteins may interact with existing proteins in unexpected ways, or otherwise alter the metabolism of the food producing organism, causing it to produce new allergens or toxins.

The food-safety issues raised by the application of gene technology to some foods have yet to be satisfactorily addressed. In some jurisdictions this has prompted calls to introduce new food labeling requirements, to help consumers identify genetically modified foods. In one recent event the sale of a gene-altered corn seed variety was halted by the owner of the variety, over concerns about human safety when the corn, not approved for human consumption, inadvertently was introduced to the human food chain (Barboza, 2000).

In a May 1999 interim statement entitled “The Impact of Genetic Modification on Agriculture, Food and Health”, the British Medical Association, Board of Science and Education, raised food allergenicity as a key area of concern about genetically modified foods, leading that organization to call for further research on the possible health risks of genetically modified foods consumption. Among other issues, was cited a March 1996 report by US researchers that a major allergen of Brazil nuts (Bertholletia excelsior) had been transferred to soybeans via genetic engineering. The allergen at issue was a high-methionine protein. As a result of this assessment, commercial interest in this transgenic soybean was abandoned (Taylor, 1997). Nonetheless, the case demonstrates the principle that allergens can be transferred between plant species.

An important facet of the overall safety assessment of genetically engineered organisms must be a thorough evaluation for possible allergenic potential. The question addressed is whether the presence of a transgene product alters the allergenic potential of a food or other products interacting with the body surface, by any mechanism. Hence, assessments must be made as to whether the presence of the transgene product unintentionally renders the crop or domesticated animal (or foods processed from them) more allergenic than a nonengineered counterpart.

It would therefore be highly desirable to provide an allergen test that (a) is simple and easy to read, (b) can identify allergenicity of a single heterologous protein, which may be present in low amounts, in a mixture of many other proteins, and (c), distinguish between allergenicity of the protein or allergenicity of the plant material in the transgenic environment. The present invention is designed to meet these needs.

### SUMMARY OF THE INVENTION

Accordingly, it is an object of the invention to provide a method for testing the allergenicity of a heterologous protein produced by genetically modified organism, e.g., a plant or animal that has been genetically modified to produce that protein. In practicing the method, the testing includes the steps of: (a) sensitizing a newborn dog from an atopic dog colony with a first extract prepared from tissue of the genetically modified plant or animal and containing a mixture of plant or animal proteins and the heterologous protein, by injecting, feeding or applying to the skin the extract into the newborn dog; (b) after a period sufficient to allow the dog to establish an immune response to the sensitizing extract, challenging the dog with the extract; (c) observing the degree of allergic response provoked; (d) if a detectable skin reaction is observed, comparing the degree of skin reaction observed with that observed by carrying out steps (a)-(c) above, but where the sensitizing step (a) or applying step (b) is carried out with a second plant or animal extract containing substantially the same proteins as the first extract but lacking the heterologous protein; and (e) if the degree of skin reaction at (c) is greater than that observed by carrying out steps (a)-(c) in accordance with step (d), identifying the heterologous protein as a potential allergen in humans.

The challenging and observing steps may include (i) applying the allergen material to a skin region of the dog and observing a local wheal reaction at the application site as the allergic response (skin test); (ii) feeding the allergen material to the dog, and observing gastrointestinal upset as the allergic response (feeding test); (iii) contacting the allergen material directly with the wall of the stomach of the dog and observing local wheal reaction at the application site as the allergic response (gastroendoscopy test); (iv) administering the allergen material by inhalation to the dog, and observing bronchial constriction as the allergic response (inhalation test); and (v) applying the allergen material with a patch immobilized on the skin and observing inflammation at the site of application.

In a preferred embodiment, the extract is obtained from a transgenic plant. In another preferred embodiment the plant is a crop plant. Preferred crop plants include corn, barley, wheat, rice, peanut, sorghum and soy.

In another embodiment, the comparison of skin reactions observed in step (d) is carried out by applying the first extract to a dog sensitized with said second extract.

In yet another embodiment, substantially no skin reaction is observed in carrying out steps (a)-(c) in step (d). Preferably, the extract can be prepared by forming a tissue powder and extracting the powder with a selected extract medium.

In still another embodiment, the method of testing the allergenicity of a heterologous protein further includes when a potential allergen is identified in step (e), repeating step (c) with the heterologous protein in purified form. In a related aspect, an organism other than the transgenic plant or animal produces the heterologous protein. In another aspect, the transgenic plant or animal produces the heterologous protein.

In one aspect, the method for testing the allergenicity of a heterologous protein further includes, when a potential allergen is identified in step (e), separating proteins in the first extract and reacting the separated proteins with an immunoglobulin obtained from the dog sensitized with the same extract, to identify whether the protein that reacts with the immunoglobulin is the heterologous protein.

In another aspect, the degree of skin reaction observed in step (c), compared with that observed in step (d) is indicative of the degree of allergenicity expected in humans.

It is another, broader, object of the invention to provide a method for testing a biological substance for allergenicity in humans, comprising the steps of: (a) sensitizing a newborn dog from an atopic dog colony with (i) at least one known allergen in humans, (ii) a non-allergen control material, and (iii) a sample containing the test substance, by injecting the allergen, control material, and test substance into the dog; (b) after a period sufficient to allow the dog to establish an immune response to the allergen: (b1) confirming that said sensitizing has provoked an appropriate immune response in the dog by challenging the dog with the known allergen and observing an allergic response in the dog, (b2) confirming that said sensitizing has not provoked an inappropriate immune response in the dog by challenging the dog with the control material and observing the absence of an allergic response in the dog, and (b3) challenging the dog with the test substance and observing the degree of allergic response provoked or no response; and (c) if an allergic response is observed in (b1) and (b3), but not (b2), identifying the test substance as a potential allergen in humans.

In a preferred embodiment, the method is used for grading the degree of allergic response produced by the test material, wherein step (a1) includes sensitizing the dog with at least two different allergens known to provoke a different degree of allergic response in humans, step (b1) includes challenging the dog with each of the at least two different known allergens, thus to determine the degree of immune response associated with the different known allergens, and in step (c) if an allergic response is observed in (b1) and (b3), but not (b2), matching the degree of response to the test allergen with one or more of the responses observed in step (b1).

In a related embodiment, the known allergens include at least three allergens selected from the group consisting of peanut extract, ragweed proteins, milk proteins, wheat proteins, and soy proteins.

For use in testing a biological substance for allergenicity in humans, a dog is provided that is (i) obtained as a newborn from an atopic dog colony and (ii) sensitized as a newborn with (a) at least one known allergen from humans, (b) a non-allergen control material, and (c) a sample containing the substance to be tested, by injecting the allergen, control material, and test substance into the dog.

In one embodiment, the dog is useful for testing allergens related to a known allergen, wherein the known allergen is a cereal, and the testing allergen is a cereal other than the known allergen.

In a related embodiment, the known allergen is a pollen, and the testing allergen is a pollen other than the known allergen. In another related embodiment, the known allergen is a nut, and the testing allergen is a nut other than the known allergen. In yet another embodiment, the dog is sensitized with at least two different allergens known to provoke a different degree of allergic response in humans.

It is still another object of the invention to provide a composition for use in sensitizing the dog, which includes a mixture of peanut proteins, ragweed proteins, milk proteins, wheat proteins, and soy proteins, in a weight/volume ratio of about 1:1:1:1:1.

These and other objects and features of the invention will be more fully appreciated when the following detailed description of the invention is read in conjunction with the accompanying tables and figures.

### BRIEF DESCRIPTION OF THE FIGURES

FIG. 1 is a time course showing the development of sensitivity of atopic dogs to preparations of peanut and ragweed.

FIG. 2 is a time course showing the development from birth sensitivity of atopic dogs to preparations of ragweed, milk, soybean and transgenic corn.

### DETAILED DESCRIPTION OF THE INVENTION

I. Definitions

The terms below, as used herein, have the following meanings, unless indicated otherwise:

As used herein, the term “atopic dog colony” refers to an inbred colony of dogs which demonstrate an IgE-mediated response to common allergens, which can be readily assessed by means of titrated tests including, but not limited to: skin tests, feeding tests, gastroendoscopy tests, inhalation tests, and dermal patch tests.

The term “dermatitis” is intended to mean any of a large family of diseases of the skin that are characterized by inflammation of the skin attributable to a variety of etiologies (Dorland's Medical Dictionary). Dermititis may be caused by inflammation to the skin including endogenous and contact dermatitis such as, but not limited to: actinic dermatitis (or photodermatitis), atopic dermatitis, chemical dermatitis, cosmetic dermatitis, dermatitis aestivalis, and seborrheic dermatitis.

As used herein, the term “transgenic plant” is intended to refer to a plant that has incorporated DNA sequences, including but not limited to genes which are perhaps not normally present, DNA sequences not normally transcribed into RNA or translated into a protein (“expressed”), or any other genes or DNA sequences which one desires to introduce into the non-transformed plant, such as genes which may normally be present in the non-transformed plant but which one desires to either genetically engineer or to have altered expression. The term also includes the progeny of said plant or plant material, including seeds and plant cells. Thus, a plant that is grown from a plant cell into which recombinant DNA is introduced by transformation is a transgenic plant, as are all offspring of that plant that contain the introduced transgene, whether produced sexually or asexually.

As used herein, the term “crop plant” means any edible or non-edible plant grown for any commercial purpose, including, but not limited to the following purposes: cosmetics, seed production, hay production, ornamental use, fruit production, berry production, vegetable production, oil production, protein production, forage production, animal grazing, golf courses, lawns, flower production, landscaping, erosion control, green manure, improving soil health, producing pharmaceutical products/drugs, producing food additives, smoking products, pulp production and wood production. Thus, crop plants include floral plants, trees, and vegetable plants.

As used herein, the term “genetic construct” refers to the DNA or RNA molecule that comprises a nucleotide sequence which encodes the desired protein and which includes initiation and termination signals operably linked to regulatory elements including a promoter and polyadenylation signal capable of directing expression in the cells into which it is introduced.

The term “sensitization” is intended for the purpose of this invention to include the induction of acquired sensitivity or of allergy. Likewise, the term “sensitize” is intended for the purposes of this invention to render sensitive or to induce acquired sensitivity.

As used herein, “heterologous DNA” or “heterologous nucleic acid” includes DNA that does not occur naturally as part of the genome in which it is present or which is found in a location or locations in the genome that differs from that in which it occurs in nature. Heterologous DNA is not naturally occurring in that position or is not endogenous to the cell into which it is introduced, but has been obtained from another cell. Generally, although not necessarily, such DNA encodes proteins that are not normally produced by the cell in which it is expressed. Heterologous DNA can be from the same species or from a different species. Heterologous DNA may also be referred to as foreign DNA. Any DNA that one of skill in the art would recognize or consider as heterologous or foreign to the cell in which is expressed is herein encompassed by the term heterologous DNA. Examples of heterologous DNA include, but are not limited to, DNA that encodes test polypeptides, receptors, reporter genes, transcriptional and translational regulatory sequences, or selectable or traceable marker proteins, such as a protein that confers drug resistance.

The terms “heterologous protein”, “recombinant protein”, “exogenous protein”, and “protein of interest” are used interchangeably throughout the specification and refer to a polypeptide which is produced by recombinant DNA techniques, wherein generally, DNA encoding the polypeptide is inserted into a suitable expression vector which is in turn used to transform a host cell to produce the heterologous protein.

That is, the polypeptide is expressed from a heterologous nucleic acid.

The term “extract” as used herein is intended to mean a concentrate of aqueous soluble plant components from the portion of the plant extracted and can be in aqueous or powdered form.

As used herein, the terms “allergic response” and “immune response” are used interchangeably and refer to an altered reactivity in response to an antigen and manifesting as various diseases, including, but not limited to, allergic rhinitis (seasonal or perennial, due to pollen or other allergens), asthma, polyps of the nasal cavity, unspecified nasal polyps, pharyngitis, nasopharyngitis, sinusitis, upper respiratory tract hypersensitivity reaction, gastrointestinal reactions and other allergies. Examples of allergies include, but are not limited to, anaphylaxis, allergic rhinitis (seasonal or perennial) or other respiratory allergy, food allergies and atopic skin reactions. Such responses can be Type I that are IgE-mediated immunologic reactions, or they can be Type II or type III that are IgA, IgG or IgM mediated reactions, or Type IV, cellular immune reactions.

The term “observe” is typically used to refer to a visual observation leading to a qualitative or quantitative determination or detection of an allergic response.

The term “organism” relates to any living entity comprised of at least one cell. An organism can be as simple as one prokaryotic cell or as complex as a animal.

“Known allergens” include, but are not limited to, milk, ragweed, wheat, barley, corn, rice, pigweed, soy, peanut, Brazil nut, English walnut, pollen extracts, dustmites, grass pollens, tree pollens (including oak and birch), mugwort, fish, shellfish, cat dander, horse dander, bee venom, wasp venom, and eggs.

As used herein, the term “microbial” includes bacteria, viruses, fungi and other microbes.

II. Method of the Invention

The invention includes, in one aspect, a method of determining the allergenicity of a heterologous protein contained in a mixture of components that express the protein. It has been discovered that even a minor allergenic component in a complex mixture of potential allergens can be detected. In addition, a determination of whether a transformed organism contains allergens resulting from the transformation process can be made. Considered below are the steps in practicing the invention.

Plants and animals and other organisms used to produce the heterologous protein can be genetically modified according to standard methods. In general, a selected nucleic acid sequence is inserted into an appropriate restriction endonuclease site or sites in a vector, which is then transformed into cells of the plant or animal. Standard methods for cutting, ligating and transforming, known to those of skill in the art, are used in constructing vectors for use in the present invention. Generally, methods for the creation of genetically modified plants, animals and other organisms for use in practicing the present invention are known to those of skill in the art. See generally, Sambrook, et al., 1989; Ausubel, et al., 1993; and Gelvin, S. B., et al., 1990, all three of which are expressly incorporated by reference, herein.

The invention contemplates that the transgenic animals of the invention can be constructed by any of the available methods including pronuclear injection and transfection of embryonic stem cells followed by blastocyst fusion to create chimeric animals. The offspring of the chimeric animals are transgenic animals. Any technique known in the art can be used to introduce the transgene which encodes the heterologous protein into animals to produce the founder lines of transgenic animals. Such techniques include, but are not limited to pronuclear microinjection (Gordon et al., 1980; Gordon and Ruddle, 1981); retrovirus mediated gene transfer into germ lines (Van der Putten et al., 1985); gene targeting in embryonic stem cells (Thompson et al., 1989; and electroporation of embryos (Lo, 1983); and sperm-mediated gene transfer (Lavitrano et al., 1989).

A. Dog Colony

The method employs a newborn dog of an atopic dog colony having a number of special characteristics. The dogs in the atopic colony are inbred, and are selected for a genetic predisposition to an allergy. The dogs may have a history of sensitivity to pollens or foods, and can be of a variety of breeds. Preferably, the dogs are spaniels or basenji dogs or mixed breed spaniel/basenji dogs. However, the dogs are not limited to these breeds. Once the dogs are produced, they can be bred, inbred, crossbred or outbred to produce further atopic colonies for use as dog models according to the present invention.

The dogs have a history of sensitivity to pollens or foods. The sensitivity can be detected using standard immunometric methods to detect serum IgE levels in the dog. These methods include, but are not limited to, IgE immunoblot enzyme linked immunosorbent assays (ELISA), radio-immunoassays (RIA), “sandwich” immunoradiometric assays (IRMA), and enzyme-labeled immunodot assays. Kits for these assays are commercially available from vendors including CMG™ (Fribourg, Switzerland) and Antibodies Inc.™ (Davis, Calif.).

Methods for performing an immunodot assay for identifying atopic dogs in accordance with the invention can be found in Ermel et al., 1997, which is expressly incorporated by reference, herein. Typically, the immunodot assay involves aliquoting food antigen extracts onto nitrocellulose strips that are then blocked with casein or ovalbumin to prevent nonspecific protein adsorption. The strips are then incubated at 4° for 18 hours in serum from the dog which has been diluted, followed by a 1 hour incubation with a primary anti-canine IgE antibody at room temperature. Bound antibodies can then be detected by incubating with anti-primary antibody immunoglobulins that are coupled to a detectable marker. Examples of suitable detectable markers include but are not limited to: enzymes, coenzymes, enzyme inhibitors, chromophores, fluorophores, chemiluminescent materials, paramagnetic metals, spin labels, and radionuclides. The strips can then be developed and quantitated by standard methods.

As seen in the present invention, dogs sensitized to an allergen from a single source (for example, wheat) can be used for testing allergens from a related source (barley or other cereals). This feature greatly broadens the use of the dog colony for testing foods or other allergenic materials.

B. Sensitizing, Challenging, and Observing Steps

B1. Sensitizing: The first step of the method involves sensitizing a newborn dog from an atopic colony with an extract by injecting into, feeding, or applying to the skin, the extract to the newborn dog. An exemplary method for sensitizing newborn dogs is given in Example 2. There are three types of extracts which can be used for sensitizing the dog.

i. Test substance/extract: The first type of extract is a test extract, which is prepared from tissue of a genetically modified plant or animal and contains a mixture of plant or animal proteins and a heterologous protein. This extract is alternatively referred to as a test substance. An exemplary method for preparing an extract from a transgenic plant is detailed in Example 1.

ii. Control substance/extract: The second type of extract is prepared from tissue of a genetically modified plant or animal and contains a mixture of plant or animal proteins, but lacks a heterologous protein. This extract may alternatively be referred to as a control substance.

iii. Known allergens: Finally, the third type of extract that can be used for sensitizing the dog is prepared from known allergens. Examples of known allergens are described in the definitions section above. Example 2 provides an exemplary method for preparing extracts of known allergens, including cow's milk, soybean, ragweed pollen, and peanut, from commercially available sources.

In one embodiment, the test extract described above is used initially to sensitize the dog. An extract is typically prepared by forming a tissue powder and extracting the powder with a selected extract medium. In one embodiment, the extract is obtained from a transgenic crop plant. Preferred crop plants are corn, barley, wheat, rice, peanut, sorghum, millet, spelt and soy.

According to one embodiment, the heterologous protein is produced by a transgenic plant or animal. According to another embodiment, the heterologous protein is produced by an organism other than a transgenic plant or animal. Examples of such organisms include fungi, bacteria, protozoa, viruses, and algae.

B2. Challenging: The second step of the method involves challenging the dog with the extract after a period sufficient to allow the dog to establish an immune response, and observing the degree of allergic response provoked or no response. The first extract used for challenging the dog is the test extract. An exemplary method showing negligible allergenicity from the extract of genetically engineered corn leaves is given in Example 4. The various methods used for challenging and observing allergic responses in the dog include skin tests, feeding tests, gastroendoscopy tests, inhalation tests and transdermal patch tests.

i. Skin test

The skin test may be used to challenge the dog by applying the allergen material to a skin region of the dog and observing local wheal formation at the application site as the allergic response. Procedures for skin tests to measure the allergic hypersensitivity reaction are described in Ermel et al., 1997, Buchanan et al., 1997, and del Val et al., 1999, each of which is expressly incorporated by reference herein. An exemplary method for performing skin tests is given in Example 3.

ii. Feeding test

The feeding test may be used to challenge the dog by feeding the allergen material to the dog, and observing gastrointestinal upset as the allergic response.

Sensitized pups challenged orally with food allergens may respond with clinical manifestations of food allergy including loose “mud-pie” diarrhea, occasional nausea and vomiting. Signs of nausea and vomiting may be acute, observed within 12 hours of food antigen exposure and may be resolved in up to about 4 days.

iii. Gastroendoscopy test

The gastroendoscopy test is used to challenge the dog by contacting the allergen material directly with the wall or injecting into the stomach of the dog and observing as the allergic response a local wheal at 3 minutes after contact and inflammation at 24 hours after contact at the application site. Procedures for gastroendoscopy tests are described in Ermel et al., 1997.

Generally, on the day before endoscopy the dogs are fed a hypoallergenic liquid maintenance elemental diet. The dogs are premedicated with atropine to minimize gastrointestinal tract secretions during the procedure. Anesthesia can be induced with Telazol (Aveco Co., Inc., Fort Dodge, Iowa) to allow intubation. Dogs are positioned in sternal recumbency for the endoscopic examinations.

The endoscopy procedure can be performed with a Pentax upper gastrointestinal tract endoscope (Pentax, Orangeburg, N.Y.) which can be fitted with an ultra miniature endoscopic videocamera. Food antigen extracts are injected into the gastric mucosa via needles passed through the biopsy channel of the endoscope.

Food allergen extracts are administered into the gastric mucosa along the ventral-lateral aspect of the greater curvature of the stomach near the confluence with the pyloric antrum. A series of dilutions of known antigens can be injected into the gastric mucosa to determine the optimal concentration for gastroscopic food sensitivity testing. A mixture of physiologic saline and glycerin can be used as a control. Approximately 5 to 10 minutes before the injections filtered 0.5% (w/v) Evans blue dye solution can be given intravenously to enhance visualization of the allergic response (0.2 ml/kg animal weight).

Gastric mucosal tissue specimens are collected before food extract and control injections with radial jaw biopsy forceps. Gastric mucosal responses are graded according to the amount of swelling, erythema, and blue patching that is observed about 3 minutes after the injection of food extract or control. The injection sites are continuously observed and videotaped for 3 minutes after each injection and biopsy specimens can be obtained immediately after the 3 minute observation period. The injection sites can be re-examined and videotaped at 15 to 30 minutes and 24 to 48 hours after the injections. Additional gastric mucosal tissue specimens are collected from the dogs 24 to 48 hours after injection. The biopsy tissue specimens can be fixed in buffered 10% formalin for histologic examination. The videotapes are reviewed and graded by persons unaware of the identity and order of the injected food antigen extracts.

iv. Inhalation test and transdermal patch test

The inhalation test may be used to challenge the dog by administering the allergen material by inhalation to the dog, and observing bronchial constriction as the allergic response. A transdermal patch may be used by applying the allergen material with a patch immobilized on the skin and observing inflammation after 24 to 72 hr at the site of application. Both of these methods are standard to one skilled in the art.

C. Analysis of reaction data

C1. Simple reaction

The third step of the method involves determining whether a detectable skin reaction has been observed after following the first and second steps described above.

C2. Qualitative analysis

In one embodiment, if a detectable skin reaction is observed, then the sensitizing, challenging and observing steps carried out above are repeated using a second plant or animal extract containing substantially the same proteins as the first extract but lacking the heterologous protein. The degree of the two skin reactions are then compared to one another.

C3. Identifying the source of allergenicity

Finally, the fourth step of the invention involves determining whether the heterologous protein is a potential allergen in humans. The protein is identified as a potential allergen in humans if the degree of skin reaction observed following sensitizing and challenging with the first extract is greater than that observed following sensitizing and challenging with the second extract which contains substantially the same proteins as the first extract but lacks the heterologous protein.

In one embodiment, when a potential allergen is identified above, the sensitizing, challenging, and observing steps are repeated with the heterologous protein in purified or partially purified form. An exemplary comparison of the skin test response to transgenic corn leaf extract and the purified protein of interest is shown in Table 2 of Example 4.

In a second embodiment, when a potential allergen is identified above, an additional step which includes separating proteins in the first extract and reacting the separated proteins with an immunoglobulin obtained from the dog sensitized with the same extract, to identify whether the protein that reacts with the immunoglobulin is the heterologous protein.

Standard methods for performing this test include, but are not limited to, enzyme linked immunosorbent assays (ELISA), radio-immunoassays (RIA), “sandwich” immunoradiometric assays (IRMA), and enzyme-labeled immunodot assays. Kits for these assays are commercially available from vendors including CMG™ (Fribourg, Switzerland) and Antibodies Inc.™ (Davis, Calif.).

Standard techniques of protein purification may be employed to separate proteins in the first extract, including: precipitation by taking advantage of the solubility of the protein of interest at varying salt concentrations, precipitation with organic solvents, polymers and other materials, affinity precipitation and selective denaturation; column chromatography, including high performance liquid chromatography (HPLC), ion-exchange, affinity, immuno affinity or dye-ligand chromatography; immunoprecipitation and the use of gel filtration, electrophoretic methods, ultrafiltration and isoelectric focusing. Each of the above-identified methods is well within the knowledge of the skilled artisan, and no undue experimentation is required to purify the proteins or epitopes of interest from any extract, using the standard methodologies outlined hereinabove, and in the literature.

Example 4 is an exemplary method showing that the invention has the ability to determine whether a transgenic protein of interest is a significant allergen. In this example, two litters of pups were sensitized to a leaf extract of genetically modified corn. At the same time, pups were sensitized to known allergenic foods, ranging from very strong (peanut) to moderately strong (milk, soy). A pollen allergen extract (giant ragweed) was also included in the sensitization regime as a reference. In parallel, one of the litters was sensitized to barley and another lifter was sensitized either to wheat or barley. The results in example 4 show that corn leaves have negligible allergenicity after being genetically engineered to contain a protein of interest, and that the protein of interest did not become allergenic.

The known allergens used to measure the relative allergenic response in the example were milk, soybean, ragweed, and peanut. However, any known allergen may be used, including those listed in the definitions section above. Likewise, any protein of interest or heterologous protein can be used including, but not limited to, enzymes, receptors, hormones, antibodies or fragments thereof, and growth factors.

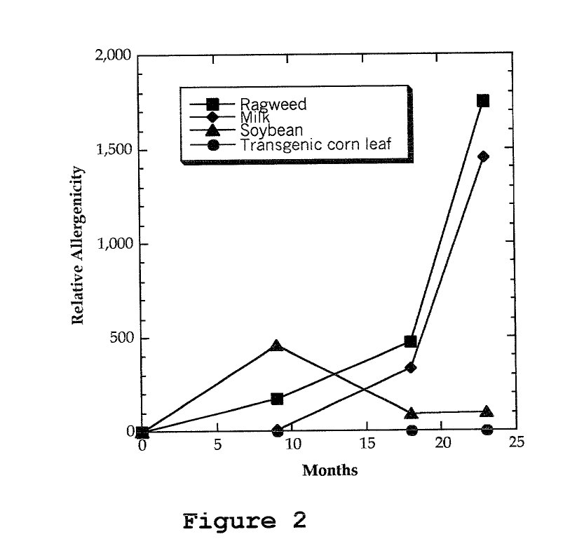

FIG. 2 is a time course showing the development from birth of sensitivity of atopic dogs to preparations of ragweed, milk, soybean and transgenic corn. Significantly, this figure, along with example 4, demonstrates that the inbred, highly allergic dogs sensitized to a non-allergenic protein do not exhibit an allergic response when challenged by that protein. Thus, by following the methods described, a determination of whether a transformed organism contains allergens resulting from the transformation process can be made.

D Use of multiple extracts

More broadly, the invention includes a method of testing a biological substance for allergenicity in humans, which includes the step of sensitizing the dog with all three extracts described in section B l above. Thus, the dog is sensitized with the test substance and at least one known allergen and one known non-allergen. After a period sufficient to allow the dog to establish an immune response, the dog is challenged with each of the extracts used for sensitization, and the allergic response is observed and analyzed as in section C above. If an allergic response is observed following a challenge with a known allergen and with the test substance, but not with the known non-allergen, then the test substance is identified as a potential allergen in humans.

In one embodiment of this broader aspect, the degree of allergic response produced by the test material is graded by sensitizing the dog with at least two different allergens known to provoke a different degree of allergic response in humans and one non-allergen, challenging the dog with each of at least two different known allergens, thus to determine the degree of immune response associated with the different known allergens, and if an allergic response is observed following the challenge with the two different allergens and with the test substance, but not with the control material, then matching the degree of response to the test allergen with one or more of the responses observed in the challenging step with the known allergens.

The known allergens for grading the response are preferably peanut extract, ragweed extract, milk proteins, wheat proteins, and soy proteins.

III. Cross reactivity of related allergens

As an important addition, a dog colony may be maintained continuously for testing allergens from a related group, or family of organisms. Example 5 provides an exemplary method of determining cross reactivity of related allergens in the atopic dog model. In this example a population of dogs sensitized to allergens was used to test the allergenicity of related plants.

From the foregoing, it can be appreciated how various objects and features of the invention are met. The testing method of the invention is effective to detect even minor allergenic components in a complex mixture of potential allergens. Accordingly, a determination of whether a transformed organism contains allergens resulting from the transformation process can be made. The following examples illustrate methods of measuring the allergenicity of a protein in accordance with the invention. The examples are intended to illustrate, but in no way limit, the scope of the invention.

### EXAMPLES

Unless otherwise indicated, all reagents and biochemicals were obtained from sources previously identified (Kobrehel et al., 1992).

### Example 1

### Test Materials

Powdered lyophilized leaf material from a transgenic corn plant was extracted with 50 mM Tris-HCl, pH 9.5, 0.1 M NaCl, 2 mM EDTA, 2 mM dithiothreitol, 1 mM 4-(2-aminoethl)-benzenesulfonyl fluoride, 1 μM leupeptin. Protein was determined using the Bradford (Bio-Rad™) Coomassie blue procedure with ovalbumin as the protein standard. Concentration of the transgene product was determined by ELISA. The corn plant expresses a transgene protein of interest (POI).

This material was found to contain on a percentage basis 0.02% transgenic protein. This extract, designated “transgenic corn leaf extract” or “transgenic preparation” was prepared at a laboratory on the East coast and shipped overnight on wet ice to the University of California, Berkeley.

### Example 2

### Animals

Immunization Schedule, Transgenic Leaf Preparation Litters.

From the original inbred colony (6 generations) of highly allergic dogs, and breeding resulted in 2 litters (7FB and 7FC, 18 pups), which were immunized with food extracts—commercial preparations of cow's milk (1:20 w/v), soybean (1:10 w/v), both from Bayer, a transgenic corn leaf extract (see above) and a commercial giant ragweed pollen extract (1:20 w/v), also from Bayer. One litter (7FB, 9 pups) was sensitized to a commercial preparation of peanut (1:10 w/v) from Meridian Biomedical. The allergenic response to the preparations was followed systematically over a two-year period.

At birth, pups were injected subcutaneously with 1 μg of each of these preparations in 0.2 ml alum. A concentration of 1 mg was based on dilution of 1 to 10 or 1 to 20 w/v of the commercial allergen extract. The real protein quantity determined by the Bradford procedure corresponded to 43 to 665 ng protein determined by the Bradford method (Bradford, 1976). At 3, 7, and 11 weeks, the animals received subcutaneously 0.5 ml of distemper/hepatitis/parvovirus live vaccine followed in 1 and 7 days after each injection with 1 μg of each allergen in alum, also injected subcutaneously. Subsequently, they were boosted every 2 months with 1 μg each of allergen in alum, then, after one year with 10 ug each of allergen in alum. At 18 months, the animals were given a distemper-hepatitis booster. Pups were injected in their axillae with a mixture containing 0.1 ml (50 to 200 ng based on Bradford) for each commercial allergen preparation or with the maximum allowable amount of the transformed leaf extract (190 ng), which contained 0.036 ng of the transgenic protein. The amount of the test protein is comparable to the level of the allergen bovine serum albumin present in milk used to sensitize the dogs. Bovine serum albumin as an allergen was consistently detected with these animals (del Val et al., 1999).

As shown below, the level of leaf extract elicited an IgE response that was independent of the transgenic protein. Allergens were combined in two groups of three allergens; one group was injected in each axilla. Commercial alum adjuvant (Mylanta, Johnson & Johnson) (0.1 ml) was added to each of three allergen mixtures. The colony of high IgE-producing atopic dogs was maintained at the Animal Resources Service, University of California, Davis (Ermel et al., 1997). The animals, representing the 7^{th} generation of the colony, were cared for according to the principles in the NIH Guide for the Care and Use of Laboratory Animals.

Immunization Schedule, Wheat, Barley, and Tree Nut Allergens.

One of the litters, sensitized to a leaf preparation from a transgenic plant, (7FB) was also sensitized to barley as described above. The third (7FC) litter of nine pups was differentially sensitized to allergens. Five were sensitized to barley (1:10 w/v, Bayer) and 4 to wheat (1:10 w/v, Bayer). Five were also sensitized to English walnut (1:20 w/v, Bayer) and 4 to Brazil nut (1:10 w/v, Bayer). The immunization schedule for this litter was as given above. Litter A not sensitized to the transgenic leaf preparation (7FA) was sensitized to milk, ragweed, wheat and soy as described above. As indicated, the response to ragweed pollen was compared to birch, oak, and pigweed pollens in ragweed-sensitive dogs. The birch, oak, and pigweed (1:20 w/v) were obtained from Bayer.

Sensitized groups.

After eighteen months, following the distemper-hepatitis vaccination, the thirteen dogs showing a low response to some or all of the allergens in skin tests were removed from the colony. After this change, the dogs showed the following sensitivities.

7FA litter (3 dogs): milk, ragweed, wheat, soy; this litter was not sensitized to transgenic leaf extract and thus can serve as a control.

7FB litter (4 dogs): milk, ragweed, barley, soy, peanut, transgenic leaf preparation.

7FC litter (7 dogs): milk, ragweed, soy, transgenic leaf preparation; (3 dogs): wheat, Brazil nut; (4 dogs): barley, English walnut.

### Example 3

### Skin Tests

Procedures for skin tests to measure the allergic hypersensitivity reaction have been described elsewhere (Ermel et al., 1997; Buchanan et al., 1997; del Val et al., 1999). In brief, 0.5% Evans blue dye was injected intravenously 5 minute prior to skin testing. Aliquots of 0.1 ml of the individual extracts were injected intradermally on ventral abdominal skin in half-log dilutions. Skin reactions were read blindly by the same experienced observer scoring two perpendicular diameters of each blue spot. Appropriate negative controls [diluted in physiological saline (10 mM Na₂HPO₄, 1.8 mM KH₂PO₄, pH 7.4, 2.7 mM KCl, 137 mM NaCl)] were included for each animal tested.

### Example 4

### Results of Transgenic Leaf Preparation Tests with Allergens of Interest

The results demonstrate that the extract from genetically engineered corn leaves has negligible allergenicity (Table 1). In Table 1 the minimum ng value represents the median amount of the preparation eliciting a wheal for the animals retaining sensitivity for the 23-month period.

As demonstrated by Part A of Table 1, only those dogs giving a response that fell in the range of 10-× or 1/10^{th} that of the median response of all dogs tested were included in the calculations. The numbers in parentheses represent the actual number of dogs used for each calculation.

As shown by Part B of Table 1, the relative skin test response of the transformed leaves extract after 23 months was approximately 1/5,000^{th} that of peanut, a very strong food allergen, to 1/700^{th} and 1/50^{th} that of the moderate food allergens milk and soybean, respectively, and 1/1900^{th} that of ragweed, a well charactized pollen allergen. These values were calculated by dividing the minimal amount of transgenic corn leaf extract giving a wheal at 23 months (65,000 ng) by the minimal amount of the indicated allergen extracts giving a wheal.

TABLE 1
Development of Allergenic Response in Dogs: Transcienic
Corn Leaf Extract vs. Known Allergens
A. Minimum ng inducing a skin response (wheal ≧ 3 mm)

| Preparation | | 9 mo | 18 mo | 23 mo |
|---|---|---|---|---|
| Peanut | Avg | 10 (4) | 22 (4) | 12 (4) |
| | Std-Dev | 10 | 0 | 12 |
| Milk | Avg | 12,594 (7) | 337 (8) | 77 (7) |
| | Std-Dev | 17,423 | 121 | 135 |
| Ragweed | Avg | 636 (9) | 237 (11) | 64 (11) |
| | Std-Dev | 716 | 454 | 76 |
| Soybean | Avg | 244 (10) | 1,195 (10) | 1,135 (10) |
| | Std-Dev | 287 | 1,908 | 1,936 |
| Transgenic | Avg | 112,000 (8) | 68,800 (7) | 64,960 (9) |
| corn leaf | Std-Dev | 0 | 53,880 | 55,821 |

The minimum ng value represents the median amount of the preparation eliciting a wheal for the animals retaining sensitivity for the 23-month period. Only those dogs giving a response that fell in the range of 10-× or 1/10^{th} that of the median response of all dogs tested were included in the calculations. The numbers in parentheses represent the actual number of dogs used for each calculation.

B. Relative Allergenicity

| Preparation | 9 mo | 18 mo | 23 mo |
|---|---|---|---|
| Peanut | 11,200 | 5,091 | 9,333 (4666) |
| Milk | 9 | 332 | 1,455 (727) |
| Ragweed | 176 | 473 | 1,750 (875) |
| Soybean | 459 | 94 | 99 (50) |
| Transgenic corn leaf | 1 | 2 | 2 (1) |

The values were calculated by dividing the minimal amount of transgenic corn leaf extract giving a wheal at 9 months (112,000 ng) by the minimal amount of the indicated allergen extracts giving a wheal.

The results show that, in the dog model, the transgenic protein in the corn leaf is not a significant allergen. This conclusion is confirmed by results shown in Table 2, Part A, which demonstrates that after 18 months exposure to the protein of interest there was no response elicited in dogs sensitized to the transgenic leaf preparation, when tested at levels ranging from 5-× to 380-× the amount present in the parent leaf extract. The quantity of the transgenic corn leaf extract found to be the lowest amount giving a wheal (29 ng) was tested. As indicated in Table 2, the protein of interest was tested using 29 ng protein (approximately 5-× POI), 2.11 μg protein (approximately 380-× POI), and 14.6 μg protein (approximately 2,500-× POI) and 49.2 μg (approximately 7,000-× POI). The number of dogs tested was 11.

Skin tests should detect the protein of interest in the transgenic leaf extract. In work using milk (del Val et al., 1999) it was revealed that 0.10 ng of the milk allergens, serum albumin, was detectable—i.e., about 1/50^{th} the level of the protein of interest tested in the experiment in Table 2, Part A. Only at a level of 7,000-× that present in the transgenic leaf extract was response observed with the protein of interest (18 month trial). Even at this level, the response was very weak and corresponded approximately to that observed with control corn leaf extract.

Relative Allergenicity Between Transgenic Corn and Purified Transgenic Protein.

A comparison of the skin test response observed after 9, 18, and 23 months for the control and genetically transformed corn leaves is also shown in Table 2. The values in Part A were calculated by dividing the minimal amount of transgenic corn leaf extract giving a wheal, for example, 112,000 ng at 9 months, by the minimal amount of the indicated allergen extracts giving a wheal. The skin test data indicate that there is no significant difference in the allergenicity of the two preparations. The results provide additional evidence that the protein of interest is not an allergen (see also Table 1, Part B). The data further indicate that the transgenic leaf does not contain allergens resulting from the transformation process.

TABLE 2
Skin Test Responses to Transgenic Corn Leaf Extract and
Different Amounts of Purified Protein of Interest (POI)
A. Minimum ng inducing a skin response (wheal ≧ 3 mm)

| Extract | | 9 mo | 18 mo | 23 mo |
|---|---|---|---|---|
| Control corn leaf | Avg | 88,107 | 28,305 | 4,446 |
| | Std-Dev | 47,412 | 41,464 | 4,947 |
| Transgenic corn leaf | Avg | 112,000 | 68,800 | 64,960 |
| | Std-Dev | 0 | 53,880 | 55,820 |
| POI equivalent to 5-X | Avg | nr | nr | nr |
| transgenic corn leaf (29 ng) | Std-Dev | — | — | |
| POI equivalent to 380-X | Avg | — | — | nr |
| transgenic corn leaf (2.1 mg) | Std-Dev | — | — | |
| POI equivalent to 2,500-X | Avg | nr | — | |
| transgenic corn leaf (14.6 mg) | Std-Dev | — | — | |
| POI equivalent to 7,000-X | Avg | — | 40,380 | |
| transgenic corn leaf (49.2 mg) | Std-Dev | — | 13,590 | |

nr = no reaction

The quantity of the transgenic corn leaf extract found to be the lowest amount giving a wheal (29 μg) was tested. As indicated, the transgenic extract was tested using 29 ng protein (approximately 5-× POI), 2.11 μg protein (approximately 380-× POI), and 14.6 μg protein (approximately 2,500-× POI) and 49.2 pg (approximately 7,000-× POI). Number of dogs tested: 11

B. Relative Allergenicity

| Preparation | 9 mo | 18 mo | 23 mo |
|---|---|---|---|
| Control corn leaf | 1.3 | 4.0 | 25.2 |
| Transgenic corn leaf | 1.0 | 1.6 | 1.7 |
| POI equivalent to 380-X transgenic corn leaf | — | — | nr |

nr = no reaction

The values were calculated by dividing the minimal amount of transgenic corn leaf extract giving a wheal at 9 months (112,000 ng) by the minimal amount of the indicated allergen extracts giving a wheal.

Course of Allergen Development to Known Allergens

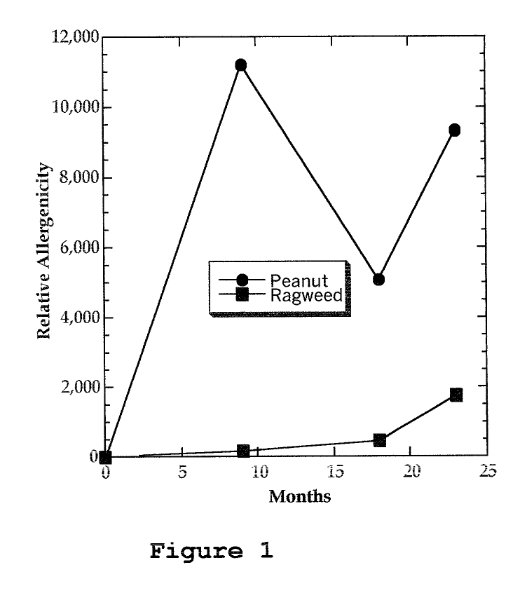

The development of the IgE response to the different allergens tested is shown in FIGS. 1 and 2. Response to peanut, by far the strongest allergen, developed rapidly and was essentially unchanged between the 9 and 23-month tests (FIG. 1). By contrast, the response to milk progressed from zero at 9 months to a very significant level at 18 and 23 months (FIG. 2). Ragweed followed a similar pattern. Soybean was different and showed the strongest response at 9 months and a weaker response at 18 and 23 months. Consistent with the data presented above, the transgenic corn leaf preparation showed essentially no response throughout the trial period (FIG. 2). The values in FIGS. 1 and 2 are based on the data in Table 1.

The results indicate that an allergen such as peanut manifests a stable IgE response earlier than the weaker allergens used in this study. Significantly, if this were the case with a test transgenic protein preparation, one could ascertain potential allergenicity for humans as well as dogs in less than one year.

### Example 5

### Cereal and Pollen Cross Reactivities

Dogs sensitized to either wheat or barley were tested for cross reactivity to the opposing preparation as well as to corn and rice. The results (Table 3) demonstrate that the dogs sensitized to wheat showed the best cross reactivity. Of 4 wheat sensitized dogs, all were also sensitive to wheat and barley, 2 were sensitive to rice, and one to corn grain. Of the 7 barley-sensitized dogs, 5 showed a significant response to barley, 4 to wheat, 3 to rice, and one to corn. In Part A the minimum ng value represents the minimal amount of the preparation inducing a wheal. In Part B, results were calculated by assuming the response to wheat in each wheat-sensitized dog and to barley in each barley-sensitized dog to be 100%. Those results used were taken from Table 3, Part A.

The data indicate that the wheat dogs are the strongest cross reactors and display very good cross reactivity with barley, some cross reactivity with rice but limited cross reactivity with corn, a grain not considered a major allergen for humans.

TABLE 3
Cross Reactivity of Cereal Allergens in
Wheat and Barley-Sensitized Dogs

| | | | | |
|---|---|---|---|---|
| Dogs | Wheat | Barley | Corn | Rice |

A. Minimum ng inducing a skin response (wheal ≧ 3 mm)

| | | | | | |
|---|---|---|---|---|---|
| Wheat | 7FA9 | 177 | 143 | 155 | 680 |
| | 7FC6 | 177 | 143 | nr | nr |
| | 7FC8 | 177 | 1,425 | 15500* | 6800* |
| | 7FC9 | 1,767 | 1,425 | 15500* | 680 |
| Barley | 7FB4 | 1,767 | 1,425 | 155 | 680 |
| | 7FB5 | 1.8 | 1.4 | nr | 68 |
| | 7FB6 | 17667* | 143 | 15500* | 68000* |
| | 7FB9 | 177 | 143 | 15500* | 680000* |
| | 7FC1 | 177 | 1,425 | nr | 680 |
| | 7FC3 | 176667* | 1,425 | 15500* | 6800* |
| | 7FC4 | 17667* | 14250* | 15500* | 68000* |

B. Relative allergenicity, %

| | | | | | |
|---|---|---|---|---|---|
| Wheat | 7FA9 | 100 | 124 | 114 | 26 |
| | 7FC6 | 100 | 124 | 0 | 0 |
| | 7FC8 | 100 | 12 | 0 | 0 |
| | 7FC9 | 100 | 124 | 0 | 260 |
| Barley | 7FB4 | 81 | 100 | 919 | 209 |
| | 7FB5 | 78 | 100 | 0 | 2 |
| | 7FB6 | 0 | 100 | 0 | 0 |
| | 7FB9 | 81 | 100 | 0 | 0 |
| | 7FC1 | 805 | 100 | 0 | 210 |
| | 7FC3 | 0 | 100 | 0 | 0 |
| | 7FC4 | 0 | 0 | 0 | 0 |

nr = no reaction
Numbers with an asterisk (*) indicating a protein concentration >5,000 ng are insignificant.
A. The minimum ng value represents the minimal amount of the preparation inducing a wheal.
B. For this table, results were calculated by assuming the response to wheat in each wheat sensitized dog and to barley in each barley-sensitized dog to be 100%. The results used were taken from A above.

Nut Allergen Cross Reactivities

The testing of the dogs sensitized to the different nuts, i.e., Brazil, peanut, and walnut, revealed some but much less cross reactivity than the cereals (Table 4). Of the 3 Brazil nut—and 4 walnut-sensitized dogs, only one walnut-sensitized dog showed significant cross reactivity to all 3 nuts; of the 4 peanut-sensitized dogs, none showed significant cross reactivity to the other nuts. The results suggest that, while there is some cross reactivity among nut allergens, the reactivity is much less than that with the cereals. This finding is consistent with the known greater taxonomic difference among the nuts relative to the cereals. In Table 4 the minimum ng value represents the minimal amount of the preparation inducing a wheal. In Part B results are calculated by assuming the response to peanut, walnut, and Brazil nut in the corresponding sensitized dogs to be 100%. The results used were taken from Table 4, Part A. The very high sensitivity to peanut is reflected in the comparison of its cross reactivity with walnut and Brazil nut in Parts A and B.

TABLE 4
Cross Reactivity of Nut Allergens in Peanut-,
Walnut- and Brazil Nut-sensitized Dogs

| | | | |
|---|---|---|---|
| Dogs | Peanut | Walnut | Brazil nut |

A. Minimum ng inducing a skin response (wheal ≧ 3 mm)

| | | | | |
|---|---|---|---|---|
| Peanut | 7FB5 | 0.002 | 2793 | 7400* |
| | 7FB4 | 0.002 | 2793 | 74 |
| | 7FB6 | 0.2 | nr | 7400* |
| | 7FB9 | 0.002 | nr | 7400* |
| Walnut | 7FC1 | 222 | 28 | 740 |
| | 7FC3 | nr | 279 | 740 |
| | 7FC4 | nr | 28 | nr |
| | 7FC5 | 2217 | 279 | 7400* |
| Brazil nut | 7FC6 | nr | nr | 74 |
| | 7FC8 | 22 | nr | 0.74 |
| | 7FC9 | nr | nr | 7.4 |

B. Relative Allergenicity, %

| | | | | |
|---|---|---|---|---|
| Peanut | 7FB5 | 100 | 0 | 0 |
| | 7FB4 | 100 | 0 | 0 |
| | 7FB6 | 100 | 0 | 0 |
| | 7FB9 | 100 | 0 | 0 |
| Walnut | 7FC1 | 12 | 100 | 4 |
| | 7FC3 | 0 | 100 | 38 |
| | 7FC4 | 0 | 100 | 0 |
| | 7FC5 | 13 | 100 | 0 |
| Brazil nut | 7FC6 | 0 | 0 | 100 |
| | 7FC8 | 3 | 0 | 100 |
| | 7FC9 | 0 | 0 | 100 |

nr = no reaction
Numbers with an asterisk (*) indicating a protein concentration >5,000 ng are insignificant.
A. The minimum ng value represents the minimal amount of the preparation inducing a wheal.
B. For this table, results were calculated by assuming the response to peanut, walnut, and Brazil nut in the corresponding sensitized dogs to be 100%. The results used were taken from A above.

Pollen Allergen Cross Reactivities.

The animals sensitized to ragweed were tested for a response to other allergenic pollens. Of the 14 dogs tested, 4 showed a significant response (relative allergenicity of at least 0.8%) to pigweed (the closest relative tested) 4 and 3 each to birch and oak tree pollens (Table 5). In Table A of Table 5 the minimum ng value represents the minimal amount of the preparation inducing a wheal, while in Part B, results are calculated by assuming the response to ragweed to be 100%. The results used were taken from Table 5, Part A.

The cross reactivity to ragweed pollen is consistent with the taxonomic relationship among these plants. It is possible that the cross-reactivity among these diverse pollens is at least in part due to profilins, ubiquitous proteins that promote acting polymerization (Valenta et al., 1992). It is known, for example, that the profilin of ragweed pollen is active with IgE elicited by another pollen, viz., mugwort (Hirschwehr et al., 1998) and that allergens in oak and birch pollens cross react with ragweed (Niederberger et al., 1998).

The cross reactivity among all the cereal, nut, and pollen allergens tested is summarized in Table 6 below.

TABLE 5
Cross Reactivity of Pollen Allergens in Dogs
Sensitized to Ragweed Pollen

| | | | | |
|---|---|---|---|---|
| Dogs | Ragweed | Birch | Oak | Pigweed |

A. Minimum ng inducing a skin response (wheal ≧ 3 mm)

| | | | | | |
|---|---|---|---|---|---|
| Ragweed | 7FA2 | 1590 | nr | nr | nr |
| | 7FA8 | 15.9 | nr | nr | nr |
| | 7FA9 | 1.59 | 9.1 | nr | 2.0 |
| | 7FB4 | 15.9 | 9.1 | 9.8 | 20.4 |
| | 7FB5 | 15.9 | 910 | 980 | 2035 |
| | 7FB6 | 15.9 | nr | nr | nr |
| | 7FB9 | 0.159 | nr | nr | 20350* |
| | 7FC1 | 0.159 | 910 | 9.8 | 20.4 |
| | 7FC3 | 15.9 | nr | nr | 20350* |
| | 7FC4 | 0.159 | nr | nr | nr |
| | 7FC5 | 1.59 | nr | nr | 2035 |
| | 7FC6 | 15.9 | nr | nr | nr |
| | 7FC8 | 0.0159 | nr | nr | nr |
| | 7FC9 | 1.59 | nr | nr | nr |

B. Relative Allergenicity, %

| | | | | | |
|---|---|---|---|---|---|
| Ragweed | 7FA2 | 100 | 0 | 0 | 0 |
| | 7FA8 | 100 | 0 | 0 | 0 |
| | 7FA9 | 100 | 17 | 0 | 78 |
| | 7FB4 | 100 | 175 | 162 | 78 |
| | 7FB5 | 100 | 1.7 | 1.6 | 0.8 |
| | 7FB6 | 100 | 0 | 0 | 0 |
| | 7FB9 | 100 | 0 | 0 | 0 |
| | 7FC1 | 100 | 1.7 | 1.6 | 0.8 |
| | 7FC3 | 100 | 0 | 0 | 0 |
| | 7FC4 | 100 | 0 | 0 | 0 |
| | 7FC5 | 100 | 0 | 0 | 0.1 |
| | 7FC6 | 100 | 0 | 0 | 0 |
| | 7FC8 | 100 | 0 | 0 | 0 |
| | 7FC9 | 100 | 0 | 0 | 0 |

nr = no reaction
Numbers with an asterisk (*) indicating a protein concentration >5,000 ng are insignificant.
A. The minimum ng value represents the minimal amount of the preparation inducing a wheal.
B. For this table, results were calculated by assuming the response to ragweed to be 100%. The results used were taken from A above.

TABLE 6
Summary of Cross Reactivity of Cereal, Nut and Pollen Allergens

| | Allergen tested for | No. of dogs | No. dogs with |
|---|---|---|---|
| Sensitization | cross reactivity | tested | significant reaction |
| Wheat | Wheat | 4 | 4 |
| | Barley | | 4 |
| | Rice | | 2 |
| | Corn | | 1 |
| Barley | Barley | 7 | 6 |
| | Wheat | | 4 |
| | Rice | | 3 |
| | Corn | | 1 |
| Peanut | Peanut | 4 | 4 |
| | Brazil nut | | 1 |
| | Walnut | | 2 |
| Walnut | Walnut | 4 | 4 |
| | Brazil nut | | 2 |
| | Peanut | | 2 |
| Brazil nut | Brazil nut | 3 | 3 |
| | Walnut | | 0 |
| | Peanut | | 1 |
| Ragweed | Ragweed | 14 | 14 |
| | Pigweed | | 4 |
| | Birch | | 3 |
| | Oak | | 3 |

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

It is claimed:

1. A method for testing the allergenicity of a heterologous protein produced by a plant or animal that has been genetically modified to produce that protein, comprising the steps of:
(a) sensitizing a newborn dog from an atopic dog colony with a first extract prepared from tissue of the genetically modified plant or animal and containing a mixture of plant or animal proteins and the heterologous protein, by injecting, feeding or applying the extract to the skin of the newborn dog,
(b) after a period sufficient to allow the dog to establish an immune response to the sensitizing extract, challenging the dog with the extract,
(c) observing the degree of allergic response provoked,
(d) if a detectable skin reaction is observed, comparing the degree of skin reaction observed with that observed by carrying out steps (a)-(c) above, but where the sensitizing step (a) or applying step (b) is carried out with a second plant or animal extract containing substantially the same proteins as the first extract but lacking the heterologous protein, and
(e) if the degree of skin reaction at (c) is greater than that observed by carrying out steps (a)-(c) in accordance with step (d), identifying the heterologous protein as a potential allergen in humans.

2. The method of claim 1, wherein said challenging and observing steps are selected from the group consisting of:
(a) applying the allergen material to a skin region of the dog and observing a local wheal reaction at the application site as the allergic response (skin test);
(b) feeding the allergen material to the dog, and observing gastrointestinal upset as the allergic response (feeding test);
(c) injecting the allergen material directly with the wall of the stomach of the dog and observing a local wheal reaction at the application site as the allergic response (gastroendoscopy test);
(d) administering the allergen material by inhalation to the dog, and observing bronchial constriction as the allergic response (inhalation test); and
(e) applying the allergen material with a patch immobilized on the skin and observing inflammation at the site of application (transdermal patch test).

3. The method of claim 1, wherein the extract is obtained from a transgenic plant.

4. The method of claim 3, wherein the plant is a crop plant selected from the group consisting of corn, barley, wheat, rice, peanut, sorghum, millet, spelt and soy.

5. The method of claim 1, wherein step (d) is carried out by applying the first extract to a dog sensitized with said second extract.

6. The method of claim 1, wherein substantially no skin reaction is observed in carrying out steps (a)-(c) in step (d).

7. The method of claim 1, wherein said extract is prepared by forming a tissue powder and extracting the powder with a selected extract medium.

8. The method of claim 1, which further includes, when a potential allergen is identified in step (e), repeating step (c) with the heterologous protein in purified form.

9. The method of claim 8, wherein the heterologous protein is produced by an organism other than the transgenic plant or animal.

10. The method of claim 8, wherein the heterologous protein is produced by the transgenic plant or animal.

11. The method of claim 1, which further includes, when a potential allergen is identified in step (e), separating proteins in the first extract and reacting the separated proteins with an immunoglobulin obtained from the dog sensitized with the same extract, to identify whether the protein that reacts with the immunoglobulin is the heterologous protein.

12. The method of claim 1, wherein the degree of skin reaction observed in step (c), compared with that observed in step (d) is indicative of the degree of allergenicity expected in humans.

13. A method for testing a biological substance for allergenicity in humans, comprising the steps of:
(a) sensitizing a newborn dog from an atopic dog colony with (a1) at least one known allergen in humans, (a2) a non-allergen control material, and (a3) a sample containing the test substance, by injecting or feeding the allergen, control material, and test substance to the dog,
(b) after a period sufficient to allow the dog to establish an immune response to the allergen:
(b1) confirming that said sensitizing has provoked an appropriate immune response in the dog by challenging the dog with the known allergen and observing an allergic response in the dog,
(b2) confirming that said sensitizing has not provoked an inappropriate immune response in the dog by challenging the dog with the control material and observing the absence of an allergic response in the dog, and
(b3) challenging the dog with the test substance and observing the degree of allergic response provoked or no response, and
(c) if an allergic response is observed in (b1) and (b3), but not (b2), identifying the test substance as a potential allergen in humans.

14. The method of claim 13, for use in grading the degree of allergic response produced by the test material, wherein step (a1) includes sensitizing the dog with at least two different allergens known to provoke a different degree of allergic response in humans, step (b1) includes challenging the dog with each of the at least two different known allergens, thus to determine the degree of immune response associated with the different known allergens, and in step (c) if an allergic response is observed in (b1) and (b3), but not (b2), matching the degree of response to the test allergen with one or more of the responses observed in step (b1).

15. The method of claim 14, wherein said known allergens include at least three allergens selected from the group consisting of peanut proteins, ragweed proteins, milk proteins, wheat proteins, and soy proteins.

16. A dog useful for testing a biological substance for allergenicity in humans,
(i) obtained as a newborn from an atopic dog colony and
(ii) sensitized as a newborn with (a) at least one known allergen from humans, (b) a non-allergen control material, and (c) a sample containing the substance to be tested, by injecting the allergen, control material, and test substance into the dog.

17. The dog of claim 16, which is sensitized with at least two different allergens known to provoke a different degree of allergic response in humans.

18. The dog of claim 16, which is useful for testing allergens related to the known allergen, wherein the known allergen is a cereal, and the testing allergen is a cereal other than the known allergen.

19. The dog of claim 18, wherein the known allergen is a pollen, and the testing allergen is a pollen other than the known allergen.

20. The dog of claim 18, wherein the known allergen is a nut, and the testing allergen is a nut other than the known allergen.

21. A composition for use in sensitizing the dog of claim 16, which includes a mixture of peanut extract, ragweed extract, milk proteins, wheat proteins, and soy proteins, in a weight/volume ratio of about 1:1:1:1:1.
